(19) United States
(12) Patent Application Publication
(10) Pub. No.: US 2007/0167510 A1
(43) Pub. Date: Jul. 19, 2007

(54) Methods for inhibiting osteoclastogenesis

(76) Inventor: Suzanne Lentzsch, Pittsburgh, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/653,011

(22) Filed: Jan. 12, 2007

Related U.S. Application Data
(60) Provisional application No. 60/759,125, filed on Jan. 13, 2006.
(60) Provisional application No. 60/853,834, filed on Oct. 23, 2006.

(51) Int. Cl. A61K 31/407

(52) U.S. Cl. 514/411

(57) ABSTRACT

Methods for the treatment of osteolytic bone disease and for the inhibition of osteoclastogenesis are disclosed. The methods utilize COX-2 inhibitors and/or inhibitors of NF-κB activation, administered to patients in an amount effective to inhibit osteoclastogenesis. Also disclosed are methods to modulate the expression of MIP-1α in a subject.

### CROSS-REFERENCE TO RELATED APPLICATIONS

This application claims benefit to U.S. Provisional Patent Application Nos. 60/759,125 filed Jan. 13, 2006 and 60/853,834 filed Oct. 23, 2006, the contents of each of which are incorporated by reference herein, in their entirety.

### FIELD OF THE INVENTION

The invention relates generally to the field of pharmacology. More specifically, the invention features methods for the treatment of osteolytic bone disease and for the inhibition of osteoclastogenesis.

### BACKGROUND OF THE INVENTION

Various publications, including patents, published applications, technical articles and scholarly articles are cited throughout the specification. Each of these cited publications is incorporated by reference herein, in its entirety. Full citations for publications not cited fully within the specification are set forth at the end of the specification.

Multiple Myeloma (MM), a cancer of plasma cells, is the second most common hematological malignancy in the United States. MM accounts for approximately 1% of all malignancies, and has an estimated incidence of 3 in 100,000, with an estimated 50,000 individuals afflicted in the United States. The disease disproportionately affects males over females, and is more common in the African American population than in the Caucasian population. In addition, the disease is more common in the elderly, the median patient age being 71. The etiology of MM is unknown, and at present, there is no cure available, although modern treatment regimens have been able to slow disease progression in many patients, and have extended survival rates to about three years post-diagnosis.

MM patients present various symptoms, including hypercalcemia, anemia, renal failure, and impaired production of non-pathological immunoglobulins. Many patients also endure persistent bone pain, especially in the pelvis, spine, ribs, and skull, which typically stems from small fractures in the bones. Indeed, the hallmark pathology of MM is increased bone destruction and development of osteolytic lesions, which make the patient more susceptible to bone fractures. Bone lesions occur in 70-80% of MM patients. (Barille-Nion S et al. (2003) Hematol. (Am. Soc. Hematol. Educ. Program) 248-78.) In addition to being a sequela of MM, osteolytic lesions also occur in connection with bone metastases of various solid tumors. Osteolytic lesions represent an extreme of a continuum of dysregulation of the normal bone remodeling process resulting from excessive bone resorption mediated by activated osteoclasts.

Bone destruction in MM is mediated by high osteoclast (OCL) activity. OCLs develop from GM-CFU derived cells, which are the committed granulocyte-macrophage progenitors, formed from precursors of myelomonocytic origin (Demulder A et al. (1993) Endocrinology 133:1978-82; Akagawa K S et al. (1996) Blood 88:4029-39; Ash P et al. (1980) Nature 283:669-70; Caux C et al. (1992) Nature 360:258-61; Reid C D et al. (1992) J. Immunol. 149:2681-88; and, Scheven B A et al. (1986) Nature 321:79-81). In MM, osteoclasts typically accumulate adjacent to patches of myeloma cells, to the exclusion of areas not invaded by the myeloma cells. (Roodman, G D (2001) J. Clin. Oncol. 19:3562-71.). This observation has led to the hypothesis that osteoclast stimulation in MM is facilitated by local processes (Callender N S et al. (2001) Semin. Hematol. 38:276-85). In fact, several candidate osteoclast-activating factors (OAFs), which are produced by myeloma plasma cells and stromal cells have been identified. (Bataille R et al. (1991) J. Clin. Invest. 88:62-6). Such OAF include IL-1, (Cozzolino F et al. (1989) Blood 74:380-7), IL-6, (Barille S et al. (2000) Eur. Cytokine Netw. 11:546-51.) and Sati H I et al. (1998) Br. J. Haematol. 101:287-95.), TNFα, (Sati H I et al. (1999). Br. J. Haematol. 104:350-7.), Hepatocyte Growth Factor (HGF), (Hjertner O et al. (1999) Blood. 94:3883-8.), lymphotoxin, (Garrett I R et al. (1987) N. Engl. J. Med. 317:526-32.), parathyroid hormone-releasing protein (PTHrP), (Uy H L et al. (1997) Cancer Res. 57:3194-9.), RANKL (Heider U et al. (2003) Clin. Cancer. Res. 9:1436-40.), and macrophage inflammatory protein (MIP)-1α (Choi S J et al. (2000) Blood. 96:671-5.).

MIP-1α has been shown to play a role in inflammation (Cook D N (1996) J. Leuk. Biol. 59:61-6.), and has also been shown to induce osteoclast formation and facilitate osteoclast chemotaxis. (Kukita T et al. (1997) Lab Invest. 76:399-406.; and Fuller K et al. (1995) J. Immunol. 154:6065-72.). In the bone marrow of MM patients, MIP-1α is overexpressed relative to healthy subjects, (Choi S J et al., 2000), and has been shown to play a role in the development of osteolytic lesions and in the accelerated bone resorption observed in MM patients. (Abe M et al. (2002) Blood. 100:2195-202.). In the same vein, neutralization of MIP-1α with an antibody was shown to inhibit excess osteoclast formation, (Choi S J et al., 2000), and antisense inhibition of MIP-1α inhibited bone destruction in a mouse model of MM (Choi S J et al. (2001) J. Clin Invest. 108:1833-41.).

The transcription factor NF-κB plays a critical role in the regulation of the cell cycle, cell adhesion, cytokine production, apoptosis, and other important cellular processes in MM. OCL formation and activity is mediated by RANKL-induced NF-κB activation (Yasuda H et al. (1998) Proc. Natl. Acad. Sci. USA 95:3597-3602). In unstimulated cells, NF-κB is localized to the cytoplasm, maintained there by its interaction with inhibitory proteins termed I kappa Bs (Karin M et al. (2000) Semin. Immunol. 12:85-98). Upon cell stimulation, NF-κB is freed from these inhibitor proteins by the action of I kappa B kinase (IKK), which phosphorylates the inhibitor proteins, targeting them for proteasome degradation.

It has been documented that IKK-γ interacts preferentially with IKKβ and is essentially required for the activation of the IKK complex through facilitating the recruitment of the IkB proteins into the IkB kinase complex (Rothwarf D M et al. (1998) Nature 395:297-300; and, Yamamoto Y et al. (2001) J. Biol. Chem. 276:36327-36). Additionally, IKK-γ maintains a stoichiometric functional IKK complex and is required by upstream signals (e.g., NIK) to activate IKK activity, serves a regulatory function for IKK activation (Ducut Sigala J L et al. (2004) Science 304:1963-7).

Prostaglandins (PG) have also been implicated in bone resorption. (Yoneda T et al. (1979) J. Exp. Med. 149:279-83.). Cyclooxygenase (COX), a key enzyme required for prostaglandin synthesis, and two isoforms (COX-1 and COX-2) have been identified. COX-2 in highly inducible form acts as a stress response gene and is responsible for the high levels of PG observed in cancer and inflammation. Inhibition of COX-2 induces anti-proliferative and pro-apoptotic effects on malignant tumors. (Nakamura S et al. (2006) Leuk Res. 30:123-35). In addition, COX-2 has been demonstrated to be a factor for osteoclast activation. (Ono K et al., (2002) J. Bone Miner. Res. 17:774-81.). Mice that fail to express COX-2 display reduced bone resorption, suggesting that COX-2 plays a role in tumor growth as well as in osteoclast activation.

Etodolac (1,8-diethyl-1,3,4,9-tetrahydropyrano-[3,4-b]indole-1-acetic acid) is a non-steroidal anti-inflammatory drug (NSAID), that inhibits both cyclooxygenase COX-1 and COX-2. (Yasui H et al. (2005) Blood. 106:706-12.). The R-enantiomer of etodolac, SDX 101, has been evaluated in phase I and II studies in B-cell malignancies. It was shown that SDX-101 induces apoptosis in MM cell lines and in freshly isolated primary MM cells. (Yasui H et al., 2005). It is believed that SDX-101 induces apoptosis by reducing the expression of cyclin D2 transcripts.

Much effort has been expended to devise effective chemotherapeutic regimens for MM patients, although such efforts have met with limited success as evidenced by the fact that the survival rates of MM patients has remained relatively unchanged over the last 20 years. Alternative therapies such as autologous or allogeneic stem cell transplantation have demonstrated treatment success and promise in a small subgroup of test patients, but it must be borne in mind that there are many MM patients whose physical condition precludes such transplantation therapies. Thus, there is a clear need for novel therapies for MM patients that may be used in addition to or in lieu of peripheral blood stem cell transplantation, as well as for treatments that can work synergistically with other therapies to improve the prognosis of patients with MM.

### SUMMARY OF THE INVENTION

The invention provides methods for treating osteolytic bone disease. In one aspect, the methods comprise administering to a subject in need of treatment for osteolytic bone disease a composition comprising a pharmaceutically acceptable carrier and a cyclooxygenase-2 (COX-2) inhibitor in an amount effective to inhibit osteoclastogenesis in the subject. The COX-2 inhibitor may be, for example, at least one of R-etodolac (SDX-101), S-etodolac, R/S-etodolac, SDX-308, celecoxib, rofecoxib, valecoxib, lumiracoxib, eltoricoxib, or an analog, homolog, conjugate, or derivative thereof. Preferably, the COX-2 inhibitor is SDX-101.

The treatment is effective for diseases and disorders characterized by increased osteoclast activity, such as, but not limited to multiple myeloma, osteoporosis, metastatic breast cancer, metastatic lung cancer, metastatic prostate cancer, infantile systemic hyalinosis, or infantile myofibromatosis. Without wishing to be bound by any particular theory of operability or mechanism of action, it is believed that the COX-2 inhibitor inhibits expression of MIP-1α.

In another aspect, the methods comprise administering to a subject in need of treatment for osteolytic bone disease a composition comprising a pharmaceutically acceptable carrier and an inhibitor of NF-κB activity in an amount effective to inhibit osteoclastogenesis in the subject. The inhibitor of NF-κB activity can modulate the activity of any constituent of the pathway for NF-κB activation. For example, but not by way of limitation, the inhibitor can inhibit dissociation of IκB, degradation of IκB, and translocation of activated NF-κB into the nucleus.

The invention also provides methods for inhibiting osteoclastogenesis in a subject by administering a composition comprising a pharmaceutically acceptable carrier and a cyclooxygenase-2 COX-2 inhibitor in an amount effective to inhibit osteoclastogenesis in the subject.

The COX-2 inhibitor may be, for example, at least one of R-etodolac, S-etodolac, R/S-etodolac, SDX-308, celecoxib, rofecoxib, valecoxib, lumiracoxib, eltoricoxib, or an analog, homolog, conjugate, or derivative thereof. Preferably, the COX-2 inhibitor is SDX-101 and/or SDX-308. The method of claim 7, wherein the COX-2 inhibitor modulates MIP-1α expression.

The invention further provides methods of modulating MIP-1α in a subject by administering a composition comprising a pharmaceutically acceptable carrier and a COX-2 inhibitor in an amount effective to modulate MIP-1α expression in the subject.

The COX-2 inhibitor may be, for example, at least one of R-etodolac, S-etodolac, R/S-etodolac, SDX-308, celecoxib, rofecoxib, valecoxib, lumiracoxib, eltoricoxib, or an analog, homolog, conjugate, or derivative thereof. Preferably, the COX-2 inhibitor is SDX-101 and/or SDX-308.

The methods of the invention are suitable for use in any animal, and are preferably used in mammals, and more preferably in humans.

### BRIEF DESCRIPTION OF THE DRAWINGS

FIG. 1 shows inhibition of growth of MM cell lines by SDX-101 and SDX-308. (A) MM cell lines MM.1S (6×10⁴/well), (B) OPM2 (3×10⁴/well) and (C)RPMI-1822 (3×10⁴/well) were incubated in 96-well culture plates (Costar, Cambridge, Mass.) in the presence of RPMI 1640 medium containing 10% FCS and SDX-101 (10, 100 μM and 1 mM) and SDX 308 (1, 10, and 100 μM) for 48 hours at 37° C. with 5% CO₂. DNA synthesis was measured by ³H— thymidine incorporation. All experiments were performed in triplicate.

FIG. 2 shows dose-dependent inhibitory effect of SDX-101 and SDX-308 on OCL formation. (A) Non-adherent bone marrow cells from healthy donor (1×10⁵) were cultured in 100 μl of α-MEM/20% horse serum, 50 ng/ml RANKL and 10 ng/ml M-CSF for 3 weeks with either 30, 50, 75, 100 μM SDX-101 or 3, 5, 7.5 and 10 μM SDX-308. After 3 weeks, the cultures were fixed with formaldehyde and stained with the 23c6 antibody. The OCLs contained three or more nuclei were scored from three independent experiments. (B) The same experiment was performed with MM bone marrow cells as A. *P<0.05 compared with control. All experiments were performed independently three times.

FIG. 3 shows that inhibition of OCL formation requires addition of SDX drugs for 3 weeks. Non-adherent cells from a healthy bone marrow donor (HBM) and a multiple myeloma bone marrow donor (MMBM) were cultured with 10 ng/ml M-CSF and 50 ng/ml RANKL for 21 days and 0.1% DMSO (control), SDX-101 (75 μM), or SDX-308 (7.5 μM) was added to the culture twice a week. Drugs were added to (A) HBM, (C) MMBM, or (F) CFU-GM colony cells either only for the first 1-week, the first 2 weeks or 3 weeks. Drugs were added to the cultures of (B) HBM, (D) MMBM, or (E) CFU-GM colony cells either only for the last week, the last 2 weeks or 3 weeks. Then the cells were fixed and stained with 23c6 antibody to detect the multinucleated mature OCLs. Data shown are the mean±STDEV of multinucleated cells (MNC) per well of at least 8 wells. Asterisks indicate significant difference from control (P<0.05). All experiments were performed independently three times.

FIG. 4 shows inhibition of bone resorption by (A) SDX-101 and (B) SDX-308, relative to controls (C). Non-adherent bone marrow cells from healthy donor (1×10⁵) were cultured in 100 μl of α-MEM/20% horse serum, 50 ng/ml RANKL and 10 ng/ml M-CSF for 3 weeks with 75 μM SDX-101 and 7.5 μM SDX-308, or 0.1% DMSO as a control. After 3 weeks, the dentin slices were fixed with formaldehyde and stained with the TRAP staining to confirm the OCL formation on the slices. Then slices were stained with hematoxylin to visualize the pit resorption areas.

FIG. 5 shows no evidence of toxic effect on hematopoietic progenitors by either SDX-101 or SDX-308. CD34⁺ cells were cultured in methylcellulose media in the presence of (A) SDX-101 (75 μM), (B) SDX-308 (7.5 μM) or DMSO (0.1%) as control for 14 days. (C-E) Numbers of the CFU colonies formed were quantified using inverted microscope. Magnifications are 10×.

FIG. 6 shows that neither SDX-101 nor SDX-308 shows toxic effect on hematopoietic progenitors or affects on osteoblast differentiation. (A) ALP activity and (B) mOG2 promoter activity: MC-42 cells were plated at a density of 5×10⁴ cells/cm2 in 35-mm plates and cultured in ascorbic acid (50 μg/ml)-containing alpha-MEM for 15 days and treated with indicated concentration of SDX-101 or SDX-308 or volume matched vehicle for 24 hours. Cells were then harvested for ALP assay and luciferase assay. ALP activity (A) and luciferase activity (B) were normalized into total protein. (C) Mineralization: MC-42 cells were grown as described in FIGS. 4C & D for 15 days. Inorganic phosphate was then added to a final concentration of 5.0 mM in the presence or absence of SDX-101 or SDX-308 or vehicle for 48 hours. Samples were then stained using the von Kossa method.

FIG. 7 shows down-regulation of secretion of cytokines (A) MIP-1α, (B) GM-CSF, (C) IFN-gamma, and (D) MIG by SDX-101 but not SDX-308. Non-adherent bone marrow cells from healthy donor were used to set up an OCL formation assay as described. During 21 days, supernatants from each half media change for control, SDX-101 and SDX-308 treated OCLs were collected and cytokine expression profile was measured as described in the Examples below.

FIG. 8 shows SDX 101 and SDX 308 inhibit RANKL-induced osteoclast formation and NF-κB activation in RAW 264.7 cells. (A) RAW cells (1×10⁴ per well) were cultured in the presence of RANKL (50 ng/ml), SDX-101, SDX-308, drug vehicle, or a combination of RANKL and drug. After 5 days, the cells were fixed and stained for TRAP activity. TRAP+ multinucleated (>3 nuclei) cells were recorded for each well. Data show the mean±SD of at least three measurements. (B) RAW cells, transiently transfected with the 3 kB-Luc-SV40 reporter gene, were treated with SDX-101, SDX-308, or vehicle in the presence or absence of RANKL. The luciferase activities were determined 8 hours after RANKL stimulation (150 ng/ml). (C and D) Raw cells were preincubated with SDX-101 or SDX-308 for 1 hour, treated with RANKL (100 ng/ml) for 30 min and then lysed. Nuclear extracts (NE) and cytoplasmic extracts (CE) were prepared using a commercial kit (Pierce, Rockford, Ill.). Phospho-p65 in CE and p65 in NE were detected by western blot assay. (E) Phospho-IκBα was detected in whole cell lysates of RAW cells by western blot analysis. β-actin served as loading control in C-E.

FIG. 9 shows that SDX-308 inhibits NF-kB activation signaling in MM cell line MM.1S cells. MM.1S M cells were preincubated with SDX-101 or SDX-308 for 1 hour, treated with TNFα (20 ng/ml) for 15 min and then lysed. NE and CE were prepared as described in FIG. 5. (A and B) Phospho-p65 and IκBα in CE and p65 in NE were detected by western blot assay. (C and D) After treatment with SDX-101 or SDX-308, MM.1S cells were stimulated by TNFα in the presence of calyculin A (50 nM) for 15 min. Phospho-IκBα (C) and phospho-IKK-γ (D) in whole cell lysates were detected using the respective antibody. B-actin served as loading control in A-D.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: MM, multiple myeloma; OCL, osteoclast; OAF, osteoclast activating factor; IL, interleukin; TNF, tumor necrosis factor; HGF, hepatocyte growth factor; MIP, macrophage inflammatory protein; PTHrp, parathyroid hormone-releasing protein; RANKL, receptor activator of nuclear factor-KB Ligand; IKK, I kappa B kinase; CSF, colony stimulating factor; COX, cyclooxygenase; PG, prostaglandin; MEM, minimal essential media; CFU, colony forming units; EMSA, electrophoretic mobility shift assay.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

“Effective amount” or “therapeutically effective amount” are used interchangeably herein, and refer to an amount of a compound, material, or composition, as described herein effective to achieve a particular biological result such as, but not limited to, biological results disclosed, described, or exemplified herein. Such results may include, but are not limited to, the treatment of osteolytic bone diseases such as multiple myeloma in a subject, as determined by any means suitable in the art.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Osteolytic bone disease” refers to any disease characterized, in whole or in part, by pathological and abnormal, dysregulated, or accelerated destruction, absorption, or dissolution of bone.

“Osteoclastogenesis” refers to the formation of or differentiation of cells into osteoclasts.

“Osteoclast” means any multinuclear cell associated with the absorption and removal of bone.

“SDX-101” refers to the R-enantiomer of etodolac having the following structure:

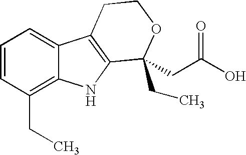

“SDX-308” refers to a tetrahydropyrano-indole structural analog of etodolac as provided by U.S. Patent. application Ser. No. 11/335,406 (Publication Number 20060167259), the contents of which are incorporated by reference herein, in their entirety and for all purposes.

Osteolytic bone disease is characterized by bone destruction and fracture, and causes pain, a loss of mobility, and an overall decrease in quality of life for a significant number of people, particularly among the aged. The bone destruction is often mediated by dysregulated or accelerated osteoclast production and stimulation. Despite increasing knowledge of the biochemical and molecular bases that underlie this pathological osteoclast production and stimulation, methods for inhibiting osteoclast production and activation, as well as methods for the treatment and prevention of osteolytic bone disease are lacking. In accordance with the present invention, it has been determined that osteoclastogenesis can be inhibited, and osteolytic bone disease can be treated through the administration of COX-2 inhibitors or of inhibitors of NF-κB activity, or through co-administration of COX-2 inhibitors and inhibitors of NF-κB activity.

Accordingly, the present invention features methods for treating osteolytic bone disease in a subject in need of such treatment, for inhibiting osteoclastogenesis in a subject, and for modulating MIP-1α expression in a subject. In some embodiments, the methods comprise administering to the subject a composition comprising a pharmaceutically acceptable carrier and a cyclooxygenase-2 (COX-2) inhibitor in an amount effective to inhibit osteoclastogenesis in the subject. In some embodiments, the methods comprise administering to the subject a composition comprising a pharmaceutically acceptable carrier and an inhibitor of NF-κB activation in an amount effective to inhibit osteoclastogenesis in the subject. In some embodiments, the methods comprise administering to the subject a composition comprising a pharmaceutically acceptable carrier and a COX-2 inhibitor and an inhibitor of NF-κB activation, each in an amount effective to inhibit osteoclastogenesis in the subject. Also featured in the invention is a method for treating osteolytic bone disease in a subject in need of such treatment, comprising modulating the expression of MIP-1α in the subject.

Non-limiting examples of the type of COX-2 inhibitors suitable for use in the methods of the present invention include RIS-etodolac, SDX-101, SDX-308 (including all enantiomers), celecoxib, rofecoxib, valecoxib, lumiracoxib, and eltoricoxib, and analogs, homologs, conjugates, or derivatives thereof. Any combination of the COX-2 inhibitors may be used as well. Weakly inhibiting COX may be likely to lessen the occurrence or severity of side effects that can occur upon inhibition of COX. One such side effect is gastrointestinal toxicity. SDX-308 is a weaker inhibitor of COX-2 than SDX-101, thus, SDX-101 is a preferred COX-2 inhibitor over SDX-308.

Any inhibitor of NF-κB activation can be used in the methods of the present invention. Suitable inhibitors will be known to those of skill in the art. SDX-308 is a stronger inhibitor of NF-κB activity than SDX-101, thus, SDX-308 is a preferred NF-κB inhibitor over SDX-101.

In the methods of the invention, subjects with osteolytic bone disease characterized by osteoclast-mediated bone destruction or loss or which are susceptible to osteoclast-mediated bone destruction or loss are identified and treated with COX-2 inhibitor or NF-κB activation inhibitor formulations as described herein for a sufficient time and with a sufficient amount to alleviate at least one sign or symptom of osteoclast-mediated bone destruction or loss.

The subject can be any animal, and preferably is a mammal such as a human, mouse, rat, hamster, guinea pig, rabbit, cat, dog, monkey, cow, horse, pig, and the like. Humans are most preferred.

The methods of the present invention are suitable for the treatment of osteolytic bone disease or other such diseases characterized by osteoclast-mediated bone destruction or loss. Non-limiting examples of osteolytic bone diseases include multiple myeloma, metastatic breast cancer, osteoporosis, Paget's disease, metastatic lung cancer, metastatic prostate cancer, infantile systemic hyalinosis, or infantile myofibromatosis. In one preferred embodiment, a subject with multiple myeloma is identified and treated with a COX-2 or NF-κB activation inhibitor formulation as described herein. In another preferred embodiment, a subject with osteoporosis or who is susceptible to osteoporosis is identified and treated with a COX-2 or NF-κB activation inhibitor formulation as described herein. In another preferred embodiment, a subject with metastatic breast cancer is identified and treated with a COX-2 or NF-κB activation inhibitor formulation as described herein. In another preferred embodiment, a subject with metastatic lung cancer is identified and treated with a COX-2 or NF-κB activation inhibitor formulation as described herein.

The invention also provides methods of inhibiting osteoclastogenesis in a subject comprising administering to the subject a composition comprising a pharmaceutically acceptable carrier and a COX-2 inhibitor or NF-κB activation inhibitor in an amount effective to inhibit osteoclastogenesis in the subject. The COX-2 inhibitor can be R/S-etodolac, SDX-101 (R-etodolac), S-etodolac, SDX-308, celecoxib, rofecoxib, valecoxib, lumiracoxib, and eltoricoxib, or any analogs, homologs, conjugates, or derivatives thereof. The NF-κB activation inhibitor can be any inhibitor suitable in the art, and preferably is SDX-308, Any combination of the COX-2 inhibitors, or combination of the COX-2 inhibitors with the NF-κB activation inhibitors can be used as well. In preferred embodiments, the COX-2 inhibitor is SDX-101. In other preferred embodiments, a combination of SDX-101 and SDX-308 is administered. The methods are suitable for use in any animal, preferably in mammals, and more preferably in humans.

Also featured in the present invention are methods for modulating the expression of MIP-1α in a subject comprising administering to the subject a composition comprising a pharmaceutically acceptable carrier and a COX-2 inhibitor in an amount effective to modulate the expression of MIP-1α in the subject. The COX-2 inhibitor can be R/S-etodolac, SDX-101 (R-etodolac), S-etodolac, celecoxib, rofecoxib, valecoxib, lumiracoxib, and eltoricoxib, or any analogs, homologs, conjugates, or derivatives thereof. Any combination of the COX-2 inhibitors may be used as well. In preferred embodiments, the COX-2 inhibitor is SDX-101. The methods are suitable for use in any animal, preferably in mammals, and more preferably in humans.

The invention also provides methods for treating osteolytic bone disease in a subject in need of such treatment, the method comprising modulating the expression of MIP-1α in the subject. The osteolytic bone disease can be any osteolytic bone disease whose etiology, physiology, or pathology is mediated in whole or in part by MIP-1α. The osteolytic bone disease can be multiple myeloma, metastatic breast cancer, osteoporosis, metastatic lung cancer, metastatic prostate cancer, infantile systemic hyalinosis, or infantile myofibromatosis, to name only a few. The methods are suitable for use in any animal, preferably in mammals, and more preferably in humans. In one preferred embodiment, the osteolytic bone disease is osteoporosis. In another preferred embodiment, the osteolytic bone disease is multiple myeloma.

Each of the various methods for treating osteolytic bone disease can be specifically adapted to treat patients within a particular ethnic group or age bracket. For example, the invention provides methods for treating osteolytic bone disease in patients having African ancestry, such as African-Americans. Such methods comprise administering to the subject having African ancestry a composition comprising a pharmaceutically acceptable carrier and at least one COX-2 inhibitor and/or at least one inhibitor of NF-κB activation in an amount effective to treat osteolytic bone disease. The COX-2 inhibitor or inhibitor of NF-κB activation can be any of those described or exemplified herein. It is preferred that the osteolytic bone disease is osteoporosis or multiple myeloma. The invention also provides methods for treating osteolytic bone disease in elderly subjects. For purposes of this invention, “elderly” refers to individuals that are at least 65 years old. Such methods comprise administering to the elderly subject a composition comprising a pharmaceutically acceptable carrier and at least one COX-2 inhibitor and/or at least one inhibitor of NF-κB activation in an amount effective to treat osteolytic bone disease. The COX-2 inhibitor or inhibitor of NF-κB activation can be any of those described or exemplified herein. It is preferred that the osteolytic bone disease is osteoporosis or multiple myeloma.

The invention further provides COX-2 inhibitors such as R/S-etodolac, SDX-101 (R-etodolac), S-etodolac, celecoxib, rofecoxib, valecoxib, lumiracoxib, and eltoricoxib, and any pharmaceutically acceptable salts, analogs, homologs, conjugates, or derivatives thereof in the manufacture of a medicament for the treatment of osteolytic bone disease and other diseases and disorders characterized by osteoclastogenesis, including without limitation, multiple myeloma, osteoporosis, metastatic breast cancer, metastatic lung cancer, metastatic prostate cancer, infantile systemic hyalinosis, and infantile myofibromatosis. In addition, the invention provides inhibitors of NF-κB activation, such as SDX-308, and any pharmaceutically acceptable salts, analogs, homologs, conjugates, or derivatives thereof in the manufacture of a medicament for the treatment of osteolytic bone disease and other diseases and disorders characterized by osteoclastogenesis, including without limitation, multiple myeloma, osteoporosis, metastatic breast cancer, metastatic lung cancer, metastatic prostate cancer, infantile systemic hyalinosis, and infantile myofibromatosis.

The COX-2 inhibitor compositions and NF-κB activation inhibitor compositions suitable for use in the present invention can be prepared in a wide variety of dosage forms according to any means suitable in the art for preparing a given dosage form. Pharmaceutically acceptable carriers can be either solid or liquid. Non-limiting examples of solid form preparations include powders, tablets, pills, capsules, lozenges, cachets, suppositories, dispersible granules, and the like. A solid carrier can include one or more substances which may also act as diluents, flavoring agents, buffering agents, binders, preservatives, tablet disintegrating agents, or an encapsulating material. Suitable solid carriers include magnesium carbonate, magnesium stearate, talc, sugar, lactose, pectin, dextrin, starch, gelatin, acacia, tragacanth, methylcellulose, sodium carboxymethyl-cellulose, polyethylene glycols, vegetable oils, agar, a low melting wax, cocoa butter, and the like. Non-limiting examples of suitable disintegrating agents include the cross-linked polyvinylpyrrolidone, agar, or alginic acid or a salt thereof such as sodium alginate. Non-limiting examples of liquid form preparations include solutions, suspensions, syrups, slurries, and emulsions. Suitable liquid carriers include any suitable organic or inorganic solvent, for example, water, alcohol, saline solution, buffered saline solution, physiological saline solution, dextrose solution, water propylene glycol solutions, and the like, preferably in sterile form.

The compositions can be formulated and administered to the subject as pharmaceutically acceptable salts. Non-limiting examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, according to means known and established in the art.

Aqueous solutions can be prepared by dissolving the active component in water and adding suitable colorants, flavors, stabilizing and thickening agents as desired. Aqueous suspensions can also be made by dispersing the finely divided active component in water with viscous material, such as natural or synthetic gums, resins, methylcellulose, sodium carboxymethylcellulose, and other well-known suspending agents.

Solid forms can be prepared according to any means suitable in the art. For example, capsules are prepared by mixing the composition with a suitable diluent and filling the proper amount of the mixture in capsules. Tablets are prepared by direct compression, by wet granulation, or by dry granulation. Their formulations usually incorporate diluents, binders, lubricants and disintegrators as well as the compound. Non-limiting examples of diluents include various types of starch, cellulose, crystalline cellulose, microcrystalline cellulose, lactose, fructose, sucrose, mannitol, kaolin, calcium phosphate or sulfate, inorganic salts such as sodium chloride and powdered sugar. Powdered cellulose derivatives are also useful. Non-limiting examples of tablet binders include starches, gelatin and sugars such as lactose, fructose, glucose and the like. Natural and synthetic gums are also convenient, including acacia, alginates, methylcellulose, polyvinylpyrrolidone and the like. Polyethylene glycol, ethylcellulose and waxes can also serve as binders.

A lubricant can be used in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances which swell when wetted to break up the tablet and release the compound, and include starches such as corn and potato starches, clays, celluloses, aligns, gums, methylcellulose, agar, bentonite, wood cellulose, powdered natural sponge, cation-exchange resins, alginic acid, guar gum, citrus pulp, carboxymethyl cellulose, and sodium lauryl sulfate. Tablets can be coated with sugar as a flavor and sealant, or with film forming protecting agents to modify the dissolution properties of the tablet. The compounds may also be formulated as chewable tablets, by using large amounts of pleasant-tasting substances such as mannitol in the formulation, as is now well-established in the art.

Also included are liquid formulations and solid form preparations which are intended to be converted, shortly before use, to liquid form preparations. Such liquid forms include solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, colorants, flavors, stabilizers, buffers, artificial and natural sweeteners, dispersants, thickeners, solubilizing agents, and the like. The compositions may be in powder form for constitution with a suitable vehicle such as sterile water, saline solution, or alcohol, before use.

The compositions can be formulated for use in topical administration. Such formulations include, e.g., liquid or gel preparations suitable for penetration through the skin such as creams, liniments, lotions, ointments or pastes, and drops suitable for delivery to the eye, ear or nose.

In some embodiments, the present compositions include creams, drops, liniments, lotions, ointments and pastes are liquid or semi-solid compositions for external application. Such compositions may be prepared by mixing the active ingredient(s) in powdered form, alone or in solution or suspension in an aqueous or non-aqueous fluid with a greasy or non-greasy base. The base may comprise complex hydrocarbons such as glycerol, various forms of paraffin, beeswax; a mucilage; a mineral or edible oil or fatty acids; or a macrogel. Such compositions may additionally comprise suitable surface active agents such as surfactants, and suspending agents such as agar, vegetable gums, cellulose derivatives, and other ingredients such as preservatives, antioxidants, and the like.

The compositions can also be formulated for injection into the subject. For injection, the compositions of the invention can be formulated in aqueous solutions such as water or alcohol, or in physiologically compatible buffers such as Hanks's solution, Ringer's solution, or physiological saline buffer. The solution may contain formulatory agents such as suspending, stabilizing and/or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol, before use.

The compositions can also be formulated in sustained release vehicles or depot preparations. Such long acting formulations may be administered by implantation (for example subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples of delivery vehicles suitable for use as carriers for hydrophobic drugs.

Administration of the compositions can be by infusion or injection (intravenously, intramuscularly, intracutaneously, subcutaneously, intrathecal, intraduodenally, intraperitoneally, and the like). The compositions can also be administered intranasally, vaginally, rectally, orally, or transdermally. Preferably, the compositions are administered orally. Administration can be at the direction of a physician.

For buccal administration, the compositions may take the form of tablets, troche or lozenge formulated in conventional manner. Compositions for oral or buccal administration, may be formulated to give controlled release of the active compound. Such formulations may include one or more sustained-release agents known in the art, such as glyceryl mono-stearate, glyceryl distearate and wax.

Compositions may be applied topically. Such administrations include applying the compositions externally to the epidermis, the mouth cavity, eye, ear and nose. This contrasts with systemic administration achieved by oral, intravenous, intraperitoneal and intramuscular delivery. Compositions for use in topical administration include, e.g., liquid or gel preparations suitable for penetration through the skin such as creams, liniments, lotions, ointments or pastes, and drops suitable for delivery to the eye, ear or nose.

Various alternative pharmaceutical delivery systems may be employed. Non-limiting examples of such systems include liposomes and emulsions. Certain organic solvents such as dimethylsulfoxide also may be employed. Additionally, the compounds may be delivered using a sustained-release system, such as semipermeable matrices of solid polymers containing the therapeutic agent. The various sustained-release materials available are well known by those skilled in the art. Sustained-release capsules may, depending on their chemical nature, release the compounds over a range of several days to several weeks to several months.

To treat a subject afflicted with osteolytic bone disease or other such disease characterized by osteoclast-mediated bone destruction or loss, a therapeutically effective amount of the composition is administered to the subject. A therapeutically effective amount will provide a clinically significant increase in healing rates in fracture repair, a decrease in osteoclast formation, a decrease in osteoclast stimulation or activation, reversal of bone loss in osteoporosis, increased bone rigidity and resistance to fracture, prevention or delay of onset of osteoporosis, and the like.

The effective amount of the composition may be dependent on any number of variables, including without limitation, the species, breed, size, height, weight, age, overall health of the subject, the type of formulation, the mode or manner or administration, or the severity of the osteolytic bone disease or other such disease characterized by osteoclast-mediated bone destruction or loss. The appropriate effective amount can be routinely determined by those of skill in the art using routine optimization techniques and the skilled and informed judgment of the practitioner and other factors evident to those skilled in the art. Preferably, a therapeutically effective dose of the compounds described herein will provide therapeutic benefit without causing substantial toxicity to the subject.

Toxicity and therapeutic efficacy of agents or compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents or compositions which exhibit large therapeutic indices are preferred. The data obtained from cell culture assays and animal studies can be used in formulating a range of dosage for use in the subject. The dosage of such agents or compositions lies preferably within a range of circulating concentrations that include the ED₅₀ with little or no toxicity. The dosage may vary within this range depending upon the dosage form employed and the route of administration utilized.

For any composition used in the methods of the invention, the therapeutically effective dose can be estimated initially from in vitro assays such as cell culture assays. For example, a dose can be formulated in animal models to achieve a circulating plasma concentration range that includes the IC₅₀ as determined in cell culture (i.e., the concentration of the composition which achieves a half-maximal inhibition of the osteoclast formation or activation). Such information can be used to more accurately determine useful doses in a specified subject such as a human. The treating physician can terminate, interrupt, or adjust administration due to toxicity, or to organ dysfunctions, and can adjust treatment as necessary if the clinical response were not adequate in order to improve the response. The magnitude of an administrated dose in the management of the disorder of interest will vary with the severity of the condition to be treated and to the route of administration. The severity of the condition may, for example, be evaluated, in part, by standard prognostic evaluation methods.

In one aspect of the inventive methods, the compositions comprise a concentration of a COX-2 inhibitor in a range of about 0.01% to about 90% of the dry matter weight of the composition. In some embodiments, the COX-2 inhibitor comprises up to about 50% of the dry matter weight of the composition. In some embodiments, the COX-2 inhibitor comprises up to about 40% of the dry matter weight of the composition. In some embodiments, the COX-2 inhibitor comprises up to about 30% of the dry matter weight of the composition. In some embodiments, the COX-2 inhibitor comprises up to about 25% of the dry matter weight of the composition. In some embodiments, the COX-2 inhibitor comprises up to about 20% of the dry matter weight of the composition. In some embodiments, the COX-2 inhibitor comprises up to about 15% of the dry matter weight of the composition. In some embodiments, the COX-2 inhibitor comprises up to about 10% of the dry matter weight of the composition. In another aspect of the inventive methods, the compositions comprise a concentration of an inhibitor of NF-κB activation in a range of about 0.01% to about 90% of the dry matter weight of the composition. In some embodiments, the inhibitor of NF-κB activation comprises up to about 50% of the dry matter weight of the composition. In some embodiments, the inhibitor of NF-κB activation comprises up to about 40% of the dry matter weight of the composition. In some embodiments, the inhibitor of NF-κB activation comprises up to about 30% of the dry matter weight of the composition. In some embodiments, the inhibitor of NF-κB activation comprises up to about 25% of the dry matter weight of the composition. In some embodiments, the inhibitor of NF-κB activation comprises up to about 20% of the dry matter weight of the composition. In some embodiments, the inhibitor of NF-κB activation comprises up to about 15% of the dry matter weight of the composition. In some embodiments, the inhibitor of NF-κB activation comprises up to about 10% of the dry matter weight of the composition.

In some embodiments, subjects can be administered the COX-2 inhibitors in a daily dose range of about 0.01 mg/kg to about 500 mg/kg of the weight of the subject. In some embodiments, subjects can be administered the inhibitor of NF-κB activation in a daily dose range of about 0.01 mg/kg to about 500 mg/kg of the weight of the subject. The dose administered to the subject can also be measured in terms of total amount of drug administered per day. In some embodiments, a subject is administered about 5 to about 5000 milligrams of SDX-101 per day. In some embodiments, a subject is administered up to about 10 milligrams of SDX-101 per day. In some embodiments, a subject is administered up to about 100 milligrams of SDX-101 per day. In some embodiments, a subject is administered up to about 250 milligrams of SDX-101 per day. In some embodiments, a subject is administered up to about 500 milligrams of SDX-101 per day. In some embodiments, a subject is administered up to about 750 milligrams of SDX-101 per day. In some embodiments, a subject is administered up to about 1000 milligrams of SDX-101 per day. In some embodiments, a subject is administered up to about 1500 milligrams of SDX-101 per day. In some embodiments, a subject is administered up to about 2000 milligrams of SDX-101 per day. In some embodiments, a subject is administered up to about 2500 milligrams of SDX-101 per day. In some embodiments, a subject is administered up to about 3000 milligrams of SDX-101 per day. In some embodiments, a subject is administered up to about 3500 milligrams of SDX-101 per day. In some embodiments, a subject is administered up to about 4000 milligrams of SDX-101 per day. In some embodiments, a subject is administered up to about 4500 milligrams of SDX-101 per day. In some embodiments, a subject is administered up to about 5000 milligrams of SDX-101 per day. In some embodiments, a subject is administered about 0.5 to about 5000 milligrams of SDX-308 per day. In some embodiments, a subject is administered up to about 1 milligrams of SDX-308 per day. In some embodiments, a subject is administered up to about 5 milligrams of SDX-308 per day. In some embodiments, a subject is administered up to about 10 milligrams of SDX-308 per day. In some embodiments, a subject is administered up to about 25 milligrams of SDX-308 per day. In some embodiments, a subject is administered up to about 50 milligrams of SDX-308 per day. In some embodiments, a subject is administered up to about 100 milligrams of SDX-308 per day. In some embodiments, a subject is administered up to about 150 milligrams of SDX-308 per day. In some embodiments, a subject is administered up to about 200 milligrams of SDX-308 per day. In some embodiments, a subject is administered up to about 250 milligrams of SDX-308 per day. In some embodiments, a subject is administered up to about 300 milligrams of SDX-308 per day. In some embodiments, a subject is administered up to about 350 milligrams of SDX-308 per day. In some embodiments, a subject is administered up to about 400 milligrams of SDX-308 per day. In some embodiments, a subject is administered up to about 450 milligrams of SDX-308 per day. In some embodiments, a subject is administered up to about 500 milligrams of SDX-308 per day.

Treatment can be initiated with smaller dosages that are less than the optimum dose of the COX-2 inhibitor, or inhibitor of NF-κB activation, followed by an increase in dosage over the course of the treatment until the optimum effect under the circumstances is reached. If needed, the total daily dosage may be divided and administered in portions throughout the day.

For effective treatment of osteolytic bone disease or other such disease characterized by osteoclast-mediated bone destruction or loss, one skilled in the art may recommend a dosage schedule and dosage amount adequate for the subject being treated. It may be preferred that dosing occur one to four or more times daily for as long as needed. The dosing may occur less frequently if the compositions are formulated in sustained delivery vehicles. The dosage schedule may also vary depending on the active drug concentration, which may depend on the needs of the subject.

The compositions utilized in accordance with the inventive methods may contain more than one COX-2 inhibitor. In some embodiments, two or more COX-2 inhibitors are administered simultaneously. In some embodiments, they are administered sequentially. The compositions can contain more than one inhibitor of NF-κB activation, which can be administered simultaneously or sequentially. The compositions can contain at least one COX-2 inhibitor and at least one inhibitor of NF-κB activation, which can be administered simultaneously or sequentially. In such embodiments, it is preferred that the COX-2 inhibitor is SDX-101, and the inhibitor of NF-κB activation is SDX-308.

The compositions of the invention for treating osteolytic bone disease or other such disease characterized by osteoclast-mediated bone destruction or loss may also be co-administered with other well known therapeutic agents that are selected for their particular usefulness against the condition that is being treated. For example, such therapeutic agents can be pain relievers, stomach antacids, compounds which lessen untoward effects of the compositions, or other known agents that inhibit osteoclast formation or activation. The compositions of the invention can also be combined with estrogens or estrogen-related compounds since estrogens are known to inhibit bone resorption. Typical estrogen and estrogen-related compounds include estradiol, progesterone, progestin, raloxifene, and analogs thereof as are well known in the art. Other compounds include but are not limited to bisphosphonates and related compounds such as, alendronate, risedronate and those set forth in U.S. Pat. No. 5,312,814, calcium supplements (Prince R1 et al. (1991) N. Engl. J. Med. 325:1189-95.), vitamin D supplements (Chapuy M C et al. (1992) N. Engl. J. Med. 327:1637-42.), sodium fluoride (Riggs B L et al. (1992) N. Engl. J. Med. 327:620-7.), androgen (Nagent de Deuxchaisnes, C., 1983, in Osteoporosis, a Multi-Disciplinary Problem, Royal Society of Medicine International Congress and Symposium Series No. 55, Academic Press, London, p. 291), and calcitonin (Christiansen, C., 1992, Bone 13 (Suppl. 1):S35).

The administration of these additional compounds may be simultaneous with the administration of the COX-2 inhibitors and/or inhibitors of NF-κB activation, or may be administered in tandem, either before or after the administration of the COX-2 inhibitors or inhibitors of NF-κB activation, as necessary. Any suitable protocol may be devised whereby the various compounds to be included in the combination treatment are administered within minutes, hours, days, or weeks of each other. Repeated administration in a cyclic protocol is also contemplated to be within the scope of the present invention.

The following examples are provided to describe the invention in greater detail. They are intended to illustrate, not to limit, the invention.

### EXAMPLE 1

### Materials and Cell and Molecular Assays Used in Subsequent Examples Chemicals

SDX-101 (R-etodolac) and SDX-308 were provided by Cephalon, Inc. (Frazer, Pa.). Both drugs were prepared in DMSO freshly for every single experiment just before use. Recombinant human receptor activator of NFκ-B ligand (RANKL) was purchased from Roche (Branchburg, N.J.), and human macrophage colony-stimulating factor (M-CSF) was obtained from R&D Systems Inc. (Minneapolis, Minn.). Alpha-Minimal essential medium (α-MEM), fetal calf serum (FCS), L-glutamine and other cell culture reagents were purchased from Invitrogen (Carlsbad, Calif.). Horse serum was obtained from Hyclone (Logan, Utah). Hypaque-Ficoll and all other chemicals were purchased from Sigma Chemical Co. (St. Louis, Mo.), unless otherwise stated.

Cells and Cell Culture:

All studies and procedures were approved by The Institutional Review Board of the University of Pittsburgh. Bone marrow cells were obtained from healthy volunteers or untreated multiple myeloma patients. Bone marrow mononuclear cells were then isolated by separation on Hypaque-Ficoll gradients according to standard protocols.

CD34⁺ cells were obtained from leukaphereses products from patients who were scheduled for autologous transplantation with enriched CD34⁺ cells. Leukaphereses products were subjected to positive selection with the Isolex 300 device. Subset analyses by FACS showed that CD34⁺ selected cells had predominantly mature phenotype (CD34⁺/CD38⁺ mean 95.5%, STDEV±5.3; CD34⁺/CD33⁺ mean 79.9%, STDEV±20.1; CD34⁺/DR⁺ mean 98.9%, STDEV±1.2). Cells were stored in media containing 10% dimethyl sulfoxide (DMSO), under liquid nitrogen.

Murine monocytic RAW 264.7 cells were obtained from the American Type Culture Collection (ATCC, Manassas, Va.) and cultured in alpha-MEM supplemented with 10% FCS, 2 mM L-glutamine, and 100 U/ml penicillin/streptomycin. This cell line expresses RANK and differentiates into tartrate resistant acid phosphatase (TRAP)-positive OCLs in the presence of bone slices or RANKL (Hsu H et al. (1999) Proc. Natl. Acad. Sci. USA 96:3540-5). MM cell lines RPMI-8226 and OPM2 were purchased from American Type Culture Collection (ATCC, Manassas, Va.). Dr. Steve Rosen kindly provided the Dex-sensitive (MM.1S) human MM cell line (Northwestern University, Chicago, Ill.). Multiple myeloma MM.1S, RPMI-8226 and OPM2 cells were cultured in RPMI-1640 medium with 10% FCS and 100 u/mL penicillin/streptomycin.

Proliferation Assay:

MM.1S cells (6×10⁴/well), RPMI-8226 cells (3×10⁴/well) or OPM2 cells (3×10⁴/well) were incubated in 96-well culture plates (Costar, Cambridge, Mass.) in the presence of RPMI 1640 medium containing 10% FCS and SDX-101 (10, 50, 100 μM and 1 mM) or SDX 308 (1, 5, 10, and 100 μM) for 48 hours at 37° C./5% CO₂. DNA synthesis was measured by ³H-thymidine incorporation (3H-TdR; NEN Products, Zaventem, Belgium). Cells were pulsed with ³H-thymidine (1 μCi/well) during the last 8 hours of culture, harvested onto glassfibre filter mats (Wallac, Gaithersburg, Md.) with an automatic cell harvester (Tomtec Harvester 960 MachIII), and counted using Wallac TriLux Beta plate scintillation counter. All experiments were performed in triplicate.

Osteoclast Formation Assay:

Non-adherent mononuclear cells (10⁵ cells/well) from either healthy donor or multiple myeloma patients, as well as purified human CD34⁺ cells, were seeded in 96-well multi-plates at 100 μl/well in α-MEM medium containing 20% horse serum, 10 ng/ml M-CSF and 50 ng/ml RANKL. SDX-101 (75 μM) or SDX-308 (7.5 μM) were added into appropriate wells for different periods (either 1, 2 or 3 weeks). Half media change was carried out twice a week.

The culture was incubated for a total of 3 weeks at 37° C. in an incubator of 5% CO₂-air. OCL formation was assessed by staining with monoclonal antibody 23c6, which recognizes the CD51/61 dimer on the osteoclast vitronectin receptor (generously provided by Michael Horton, Rayne Institute, Bone and Mineral Center, London, UK), using a Vectastatin-ABC-AP kit (Vector Laboratories, Inc. Burlingame, Calif.). The 23c6⁺ multinucleated osteoclasts containing 3 or more nuclei per osteoclast were scored using an inverted microscope. For RAW 264.7 cells, OCL differentiation assay was carried out in 96-well plates. The cells (4×10³/well) were treated with 50 ng/ml RANKL for 5 days, in the absence or presence of SDX-101 (75 μM) or SDX-308 (7.5 μM). Half media change was performed every 2-3 days by replacing with fresh medium containing RANKL with or without tested drugs. Cells were fixed and stained with TRAP-staining kit (Sigma-Aldrich) according to the manufacturer's instructions. TRAP-positive multinuclear cells (>3 nuclei) were counted under the inverted microscope.

Osteoclastic Bone Resorption on Dentin Slices:

Bone marrow cells from healthy donor (2×10⁵ cells/well) were seeded on whale dentin slices in 96-well multi-plates at 100 μl/well in α-MEM medium containing 20% horse serum. In addition, 10 ng/ml M-CSF and 50 ng/ml RANKL were added. Half media changes were performed twice a week with fresh media.

SDX-101 (75 μM) or SDX-308 (7.5 μM) were added to the well every time half media change was carried out. Culture was continued for 3 weeks at 37° C. in an incubator of 5% CO₂-air. After 3 weeks, dentin slices were stained with TRAP staining to confirm the osteoclast formation. Then, bone resorption lacunae were stained with hematoxylin. Pit area was quantified by using the public domain NIH ImageJ program. Fixed small representative areas on dentin slices selected and the mean resorption areas were determined.

Colony Assays:

CD34⁺ cells were plated in quadruplicate 35-mm plastic culture dish (15×10² cells/per dish) in 1 ml methylcellulose media (Methocult GF H4434, StemCell Technologies) and cultured in the presence of SDX-101 (75 μM), SDX-308 (7.5 μM) or DMSO (0.1%) as control. Methocult GF H4434 contained the following hematopoietic growth factors: recombinant human (rh) Erythropoietin 3 U/ml, rh SCF 50 ng/ml, rh granulocyte-macrophage colony-stimulating factor (GM-CSF) 10 ng/ml, and rh IL-310 ng/ml as well as Methylcellulose (1%), Fetal Bovine Serum 30%, Bovine Serum Albumin (1%), 2-Mercaptoethanol (10⁴M), and L-glutamine (2 mM). Cells were incubated in at 37° C., 5% CO₂ with 95% humidity for 14-16 days. Formation and relative distribution of burst forming unit-erythroid (BFU-E), colony forming unit-macrophages (CFU-M) and colony forming unit-granulocyte, macrophage (CFU-GM) colonies were evaluated by scoring under an inverted microscope. Each colony experiment was carried out in triplicate.

Osteoblast Differentiation Assays

MC-42 cells, which contain a stably transfected 1.3-kb mOG2 (mouse osteocalcin gene 2) promoter driving expression of a firefly luciferase gene, were plated in 35-mm plates and cultured in alpha-MEM containing ascorbic acid (50 μg/ml) for 15 days and treated with SDX-101 (75 μM) or SDX-308 (7.5 μM) for 24 hours. Cells were then harvested for luciferase and ALP (alkaline phosphatase) activity assay. Luciferase and ALP activity were normalized to total protein content of the sample (Xiao G et al. (1997) Mol. Endocrinol. 11:1103-13). To determine the effect of SDX-101 and SDX-308 on the mineralization potential of cultures, MC-42 cells were grown as described above for 15 days. Inorganic phosphate was then added to a final concentration of 5.0 mM in the presence or absence of previously stipulated concentrations of SDX-101 or SDX-308 for 48 hours. Samples were then stained using the Von Kossa method (Xiao G et al. (1998) J. Biol. Chem. 273:32988-94).

Quantification of Cytokine, Chemokine and Growth Factor Released from Osteoclasts:

Supernatants from each half media change during 3 weeks osteoclast formation assay from control, SDX-101 treated, and SDX-308 treated cultures were used to measure the expression levels of cytokine, chemokine, and growth factors released into supernatants of long-term human bone marrow cultures from developing osteoclast. Supernatants collected on day 5, 9, 12, 16 and 21 were analyzed for expression of IL-1α, IL-1β, IL-2, IL-3, IL-4, IL-5, IL-6, IL-7, IL-8, IL-9, IL-10, IL-13, IL-15, IL-17, IL-12p40, IFNα, IFNγ, TNF-α, TNFR-I, TNFR-II, G-CSF, GM-CSF, MCP-1, MIP-1α, MIP-1β, MIG, IP-10, EOTAXIN, RANTES, DR5, EGF, VEGF, FGF-b, and HGF by Bio-plex Cytokine Assay (BIO-RAD Laboratories, USA).

DEAE Dextran-Mediated Transfection of NF-κB Luciferase Reporter Gene

To determine the NF-κB activation in mouse osteoclast cell line RAW 264.7, cells were transient transfected with a NF-κB luciferase reporter gene. The 3 kB-Luc-SV40 plasmid, which was kindly provided by Dr. Xu (University of Western Australia, Perth), contains three NF-κB binding sites from the interferon gene upstream of the luciferase coding region. 16 0.4 μg of plasmid was directed into RAW 264.7 cells (2×10⁶) using DEAE-Dextran method. Transfected cells were seeded in 24-well plates (10⁵/well) in 10% FCS for 36 hours before treatment with SDX-101 or SDX-308 for 1 hour, followed by RANKL stimulation (150 ng/ml) for another 8 hours. After cell harvest, the cells were lysed and the luciferase activity was assayed with Promega Luciferase Assay System (Promega, Madison, Wis.).

Western Blot Analysis:

To evaluate how SDX-101 and SDX-308 affect protein expression levels of transcription factors (NFATc1, PU.1 and c-fos), total cell lysates were prepared on day 7, day 14 and day 21 from developing osteoclasts. Non-adherent mononuclear cells (2×10⁵ cells/well) from normal bone marrow were seeded in 48-well multi-plates at 400 μl/well in α-MEM medium containing 20% horse serum, 10 ng/ml M-CSF and 50 ng/ml RANKL. Both compounds were added into appropriate wells at 75 μM for SDX-101 and 7.5 μM for SDX-308 concentrations for 3 weeks every time half media change performed.

Cells were harvested, washed three times with phosphate buffered saline, and lysed with buffer containing (NP-40 1%, DTT 0.5 mM, sodium orthovanadat 1 mM, aprotinin 1 μg/mL, sodium fluoride 50 μM, phenylmethylsulfonyl fluoride 500 μM). The protein concentrations of the lysates were determined with the Bradford assay (Sigma) according to manufacturer's recommendations. Approximately 50 ng/ml protein from each condition was loaded onto 10% SDS-PAGE, and electrophoresis was carried out at 60 mA for 1 hour, followed by electro transfer onto a nitrocellulose membrane at 100 V for 1 hour. The membrane was pre-blotted with 5% dry milk (Bio-Rad Laboratories) in Tris-buffered saline (50 mM Tris-HCl and 150 mM NaCl, pH 7.5) at room temperature for 1 h. Mouse anti-human monoclonal antibody was used to detect NFATc1 protein (Santa Cruz Biotechnology Inc, CA), and rabbit anti-human polyclonal antibody for c-fos protein (Abcam, Cambridge, Mass.). Rabbit anti-PU. 1 antibody (Santa Cruz Biotechnology Inc, CA) and rabbit anti-RANK (Chemicon International) were tested for the expression levels. Bands were visualized by the enhanced chemiluminescence Western blotting detection system (Amersham Biosciences Inc., Piscataway, N.J.). A rabbit polyclonal GAPDH antibody (Santa Cruz) was used as loading control.

For detection of p65 translocation, cellular nuclear and cytoplasmic extracts were prepared with Nuclear and Cytoplasmic Extraction Reagents (Pierce, Rockford, Ill.). Cell lysates (20-40 μg) were subjected to 10% sodium dodecyl sulfate-polyacrylamide gel electrophoresis and transferred to PVDF membrane (Millipore, Danvers, Mass.). The blots were probed with anti-p65, anti-phospho p65, anti-IκB-α, anti-phospho IκB-α, or anti-phospho IKK-γ (all from Cell Signaling, Beverly, Mass.), and beta-actin antibody (Amersham, Piscataway, N.J.). Immune complexes were detected using enhanced chemiluminescence (Amersham).

Statistical Analyses:

Each experiment was repeated at least 3 times, and all quantitative data are presented as mean±S.D. Statistical differences were determined by Students' t-test. The results were considered as significantly different for P<0.05.

### EXAMPLE 2

### SDX-308 Exhibits 10-Fold Stronger Inhibition of MM Cell Growth Relative to SDX-101

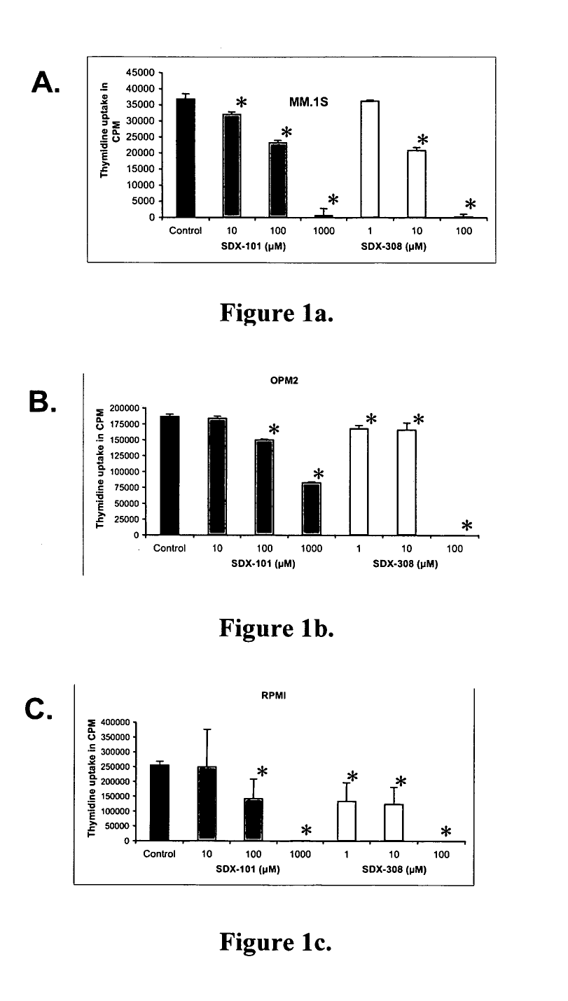

To measure the effect of SDX-101 and SDX-308 on proliferation of multiple myeloma cell lines, different multiple myeloma cell lines were evaluated: MM.1S, RPMI-8266 and OPM2 were treated with SDX-101 or SDX-308 at 1, 10, and 100 μM for 48 hours. The DNA synthesis measured by thymidine incorporation was found to be significantly inhibited at concentrations of 10 μM for SDX-101 in MM.1S cells (FIG. 1A). OPM2 and RPMI-1822 cells showed significant inhibition at 100 μM for SDX-101 (FIGS. 1B and 1C). In contrast, SDX-308 showed a 10- to 100-fold greater anti-proliferative activity, significantly inhibiting (P<0.01) MM growth at 1 μM in OPM2 and RPMI cells and at 10 μM in MM.1S cells in comparison to control treatment (FIG. 1 A-C).

### EXAMPLE 3

### SDX-101 and SDX-308 Demonstrate Dose-Dependent and Early Inhibition of Osteoclast Formation

The effect SDX-101 and SDX-308 on OCL formation was tested using human non-adherent mononuclear bone marrow cells from healthy donors and untreated multiple myeloma (MM) patients. A dose of 50 ng/ml RANKL and 10 ng/ml M-CSF was used to stimulate the development of large number of multinucleated OCL. Compounds were added to the cultures twice a week at the time of a half-media change for the duration of the 21-day assay. SDX-101 and SDX-308 produced a dose-dependent inhibition of RANK-L/M-CSF-induced human osteoclastogenesis both in cultures from healthy donor and MM patients.

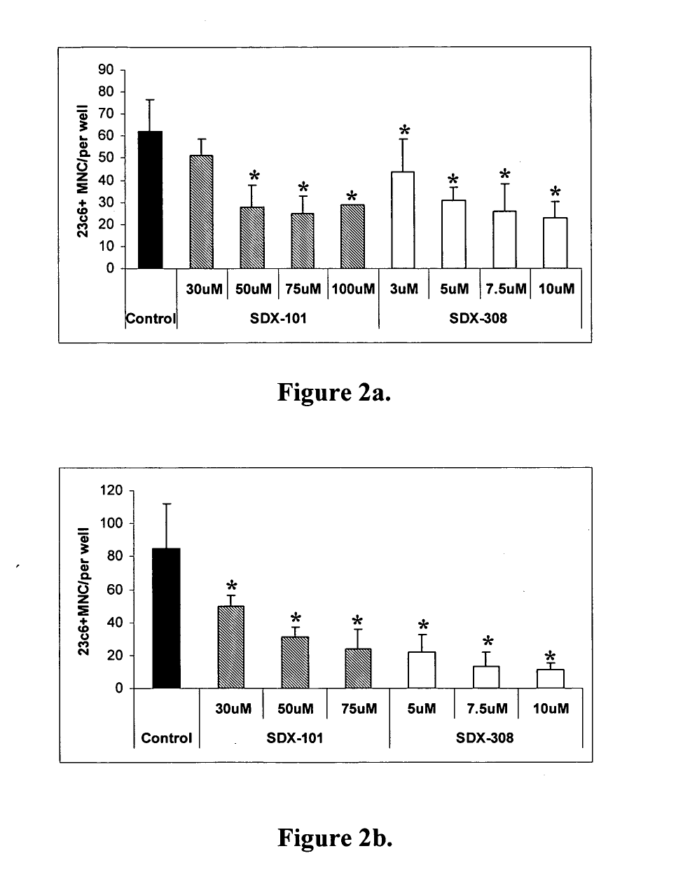

Each drug was tested at several doses, which ranged from 30 to 100 μM for SDX-101, and 3 to 10 μM for SDX-308 (FIGS. 2A and B). In the case of SDX-101, all doses over 50 μM tested exhibited statistically significant inhibition of OCL formation (FIG. 2A). Surprisingly, SDX-308 showed 10-fold stronger inhibition at all doses tested as compared to SDX-101 (FIG. 2B), and inhibited OCL formation at 3 μM and 5 μM. The same results were observed in OCL formation assays using MM mononuclear bone cells. Based on these results, a dose of 75 μM SDX-101 and 7.5 μM SDX-308 were used in the assays for the rest of the project. No cell death was observed during the 3 week OCL formation assay at any doses tested of either drug. This result indicates that both drugs are not toxic to the bone marrow cells.

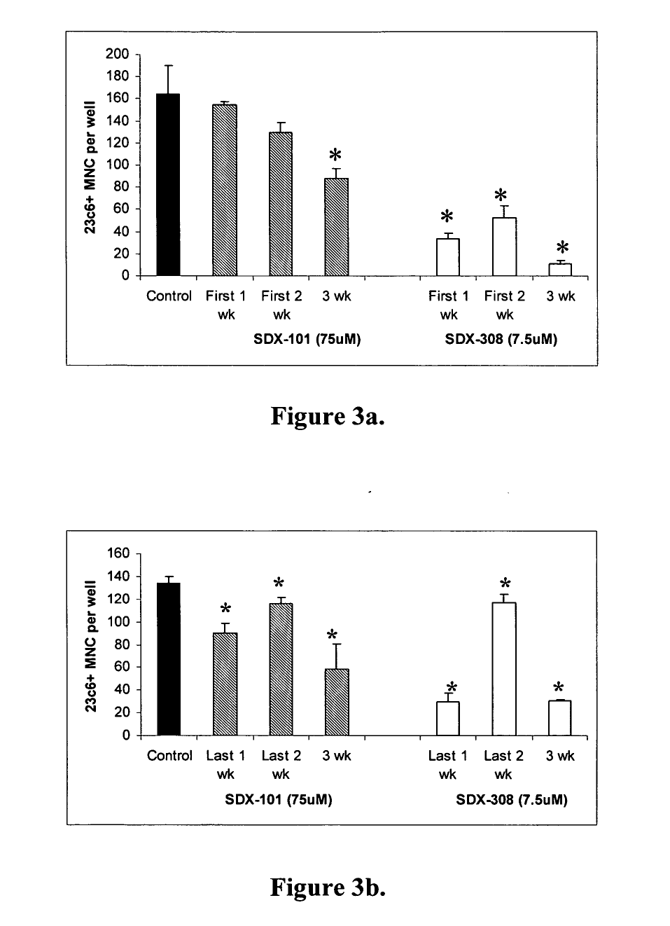

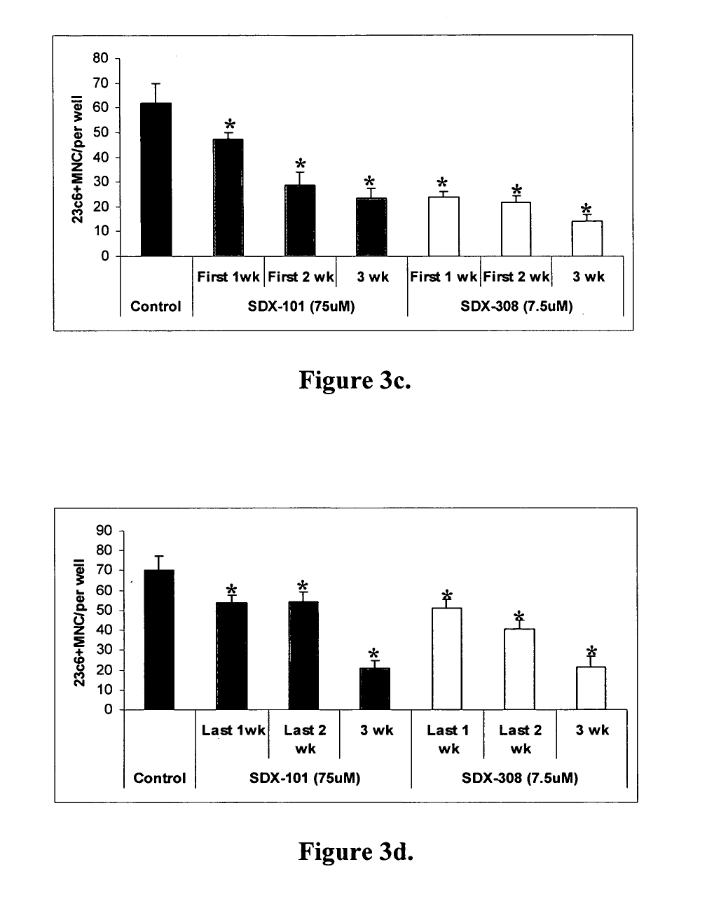

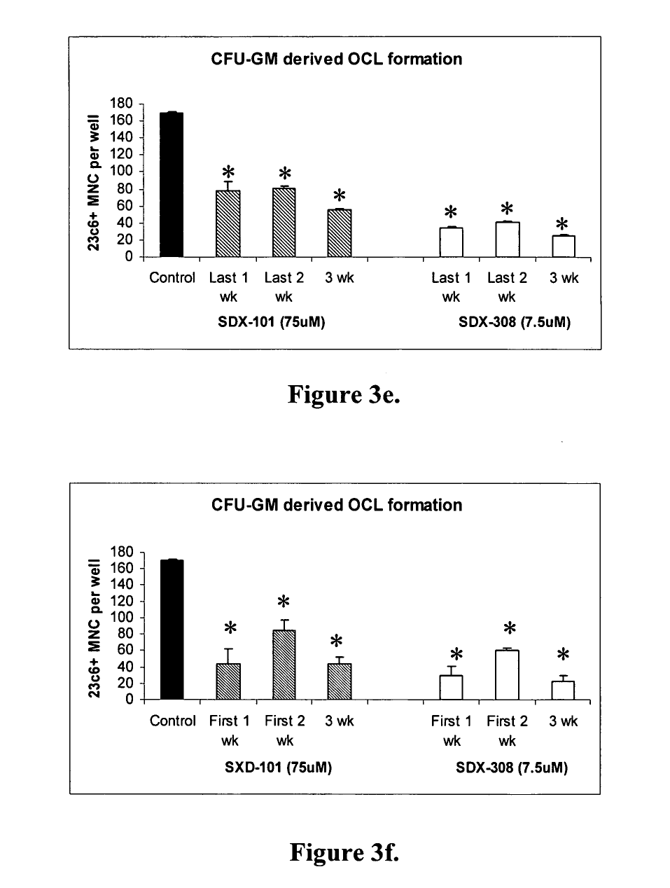

A time course experiment for OCL formation assay was performed on cells from healthy donors and MM patient in order to resolve the question of whether inhibition of OCL formation is an early event or late event. Five groups of human bone marrow cultures were treated with SDX-101 or SDX-308: (1) treatment for the entire 3 weeks (2) treatment for only the last 2 weeks (no drug treatment during the first week), (3) treatment for only the first 2 weeks (no drug treatment during the last week) (4) treatment for only the last week of the culture (no drug treatment during the first or second weeks), or (5) treatment for only the first week of the culture. As revealed in FIG. 3-A, B, C, D, E, and F, inhibition of OCL formation was significantly higher when drug was added during the entire 3 weeks of the assay. In contrast, when drug treatment was carried out for only the first or last one or two weeks of the assay, the inhibitory effect was less significant relative to exposure to the full three week period, indicating that SDX derivatives may not act preferentially on a particular stage of OCL development.

To examine whether an early stage of osteoclastogenesis is affected by SDX-101 or SDX-308, CFU-GM colonies were isolated from colony CFU-GM assays. Cells from CFU-GM colonies are osteoclast precursors. Osteoclast formation assays were set up with purified CFU-GM cells and cultured for 21 days. Drugs were added either for the first 1, 2, or all 3 weeks or for the last 1, 2, or all 3 weeks. The results show that both SDX-101 and SDX-308 do not act by inhibiting osteoclast progenitor development since the usage of osteoclast progenitor cells to set up osteoclast formation assay also led to significantly decreased osteoclast numbers. In addition, adding SDX-101 and SDX-308 at certain time points (first 1, 2 or 3 weeks or last 1, 2 or 3 weeks) showed no differences in osteoclast inhibition suggesting that these drugs do not act on certain stages of osteoclast development (FIG. 3 E, F).

### EXAMPLE 4

SDX-101 and SDX-308 Inhibit Osteoclastic Bone Resorption

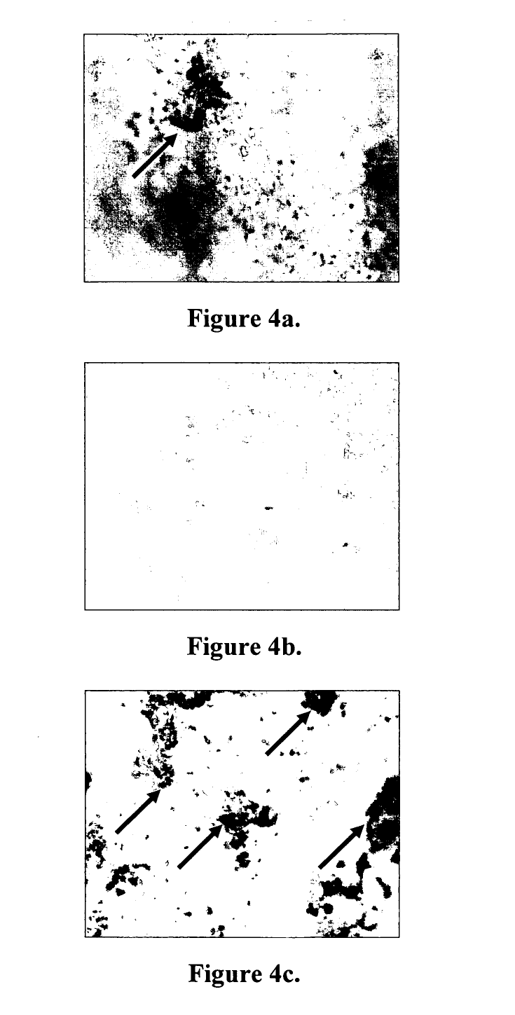

Because OCL formation is significantly inhibited by SDX-101 and SDX-308, a bone resorption assay was performed to confirm that the inhibitory effect of SDX-101 and SDX-308 is accompanied by abrogation of bone resorption activity of mature OCLs. Consistent with the results showing significant inhibition of OCL formation by SDX-101 treatment (75 μM), bone resorption was also inhibited significantly (measured bone resorption area 1.3 vs. 8.6 mm² for untreated control group) (FIG. 4A), relative to the control (FIG. 4C). Surprisingly, bone resorption resulted in complete inhibition with SDX-308 (7.5 μM) (FIG. 4B).

### EXAMPLE 5

### SDX-101 and SDX-308 Exhibit No Toxic Effects on Hematopoietic Precursors

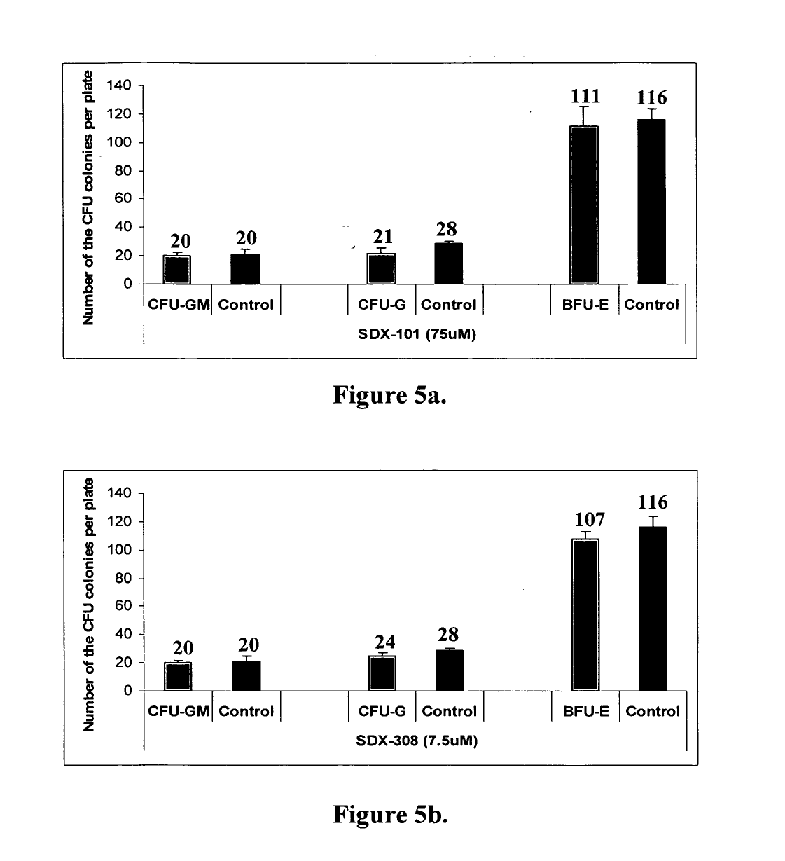

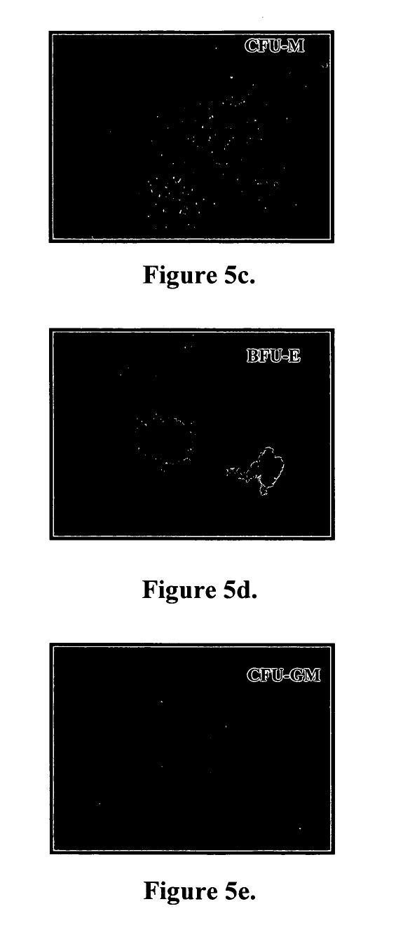

The effect of SDX-101 and SDX-308 on differentiation of hematopoietic progenitors into colony forming unit (CFU) was analyzed. Purified, human CD34+cells were embedded into the methylcellulose culture, and drugs were added only once at 3 different concentrations (30, 50 and 75 μM of SDX-101) and (1, 5, 7.5 μM of SDX-308), and incubated for 14 days. Examination of the number of colonies and the colony phenotypes was carried out using an inverted microscope. This analysis revealed that none of the doses tested for both SDX derivatives interferes with the hematopoietic lineage commitment. As shown in FIG. 5 A and B, the number of the CFU colonies formed under treatment of either SDX-101 or SDX-308 was not significantly different as compared to untreated cells, indicating that both drugs are not toxic the hematopoietic progenitors. Colony forming units were quantified by microscopy, examples of which are shown in FIG. 5C-E.

### EXAMPLE 6

### SDX-101 and SDX-308 Have No Inhibitory Effect on Osteoblast Differentiation

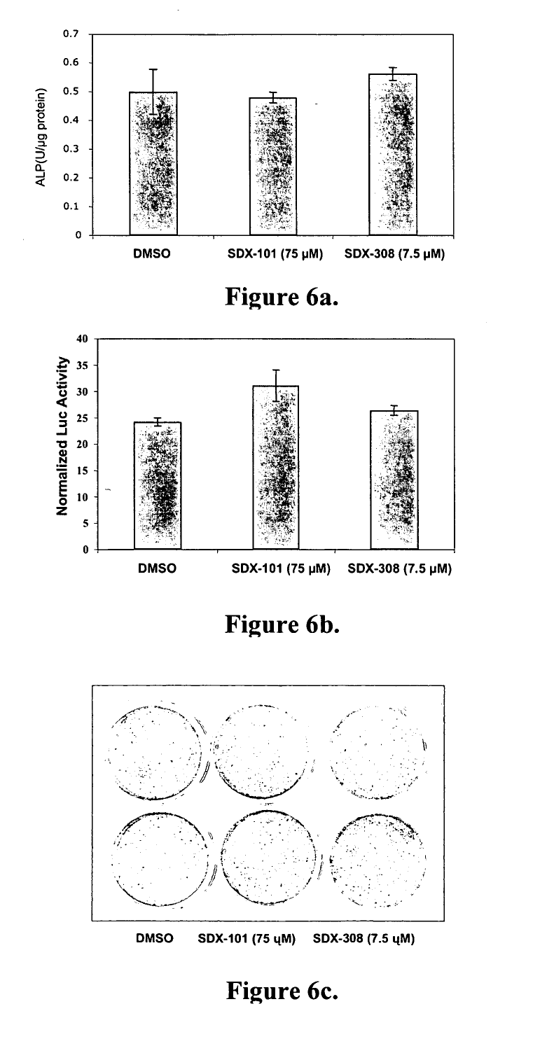

To determine whether SDX-101 or SDX-308 affect osteoblast differentiation, three experiments were carried. First the question of whether these two drugs could inhibit alkaline phosphatase activity (ALP), an osteoblast differentiation marker, in MC3T3-E1 pre-osteoblastic cells (MC-42), was investigated. As shown in FIG. 6A, treatment of MC-42 with the drugs at concentrations that significantly inhibit OCL differentiation (SDX-101 75 μM and SDX-308 7.5 μM) did not decrease the ALP activity. Second, whether the drugs could inhibit the osteoblast-specific mOG2 (mouse osteocalcin gene 2) promoter activation in differentiated MC-42 cells was determined. MC-42 cells contain a stably transfected 1.3-kb mOG2 promoter driving expression of a firefly luciferase gene and luciferase expression which closely follows levels of endogenous osteocalcin mRNA. Cells were treated with the compounds for 24 hours and then harvested for luciferase and protein assay. As shown in FIG. 6B, the mOG2 promoter activity was not inhibited by either SDX-101 or SDX-308. Third, as shown in FIG. 6C, the drug treatment did not inhibit mineralization of osteoblasts as measured by von Kossa staining. Taken together, these data suggest that neither SDX-101 nor SDX-308 effects osteoblast differentiation at concentrations that inhibit OCL differentiation and function.

### EXAMPLE 7

### SDX-101 and SDX-308 Induces a Bone Marrow Cytokine Milieu Resulting in an Inhibition of Osteoclast Formation

Hematopoiesis and osteoclastogenesis are regulated by cytokines that bind to lineage specific receptors and thereby affect lineage fate. To investigate whether the activity of SDX-101 and SDX-308 on osteoclastic cells might involve the production of specific cytokines, osteoclast formation assays were treated with SDX-101 and SDX-308 for three weeks. Supernatants were analyzed by Bio-Rad® cytokine arrays for the regulation of the following cytokines: for the regulation of the following cytokines: IL-Iα, IL-1β, IL-2, IL-3, IL-4, IL-5, IL-6, IL-7, IL-8, IL-9, IL-10, IL-13, IL-15, IL-17, IL-12p40, IFNα, IFNγ, TNF-α, TNFR-I, TNFR-II, G-CSF, GM-CSF, MCP-1, MIP-1α, MIP-1β, MIG, IP-10, EOTAXIN, RANTES, DR5, EGF, VEGF, FGF-b, and HGF.

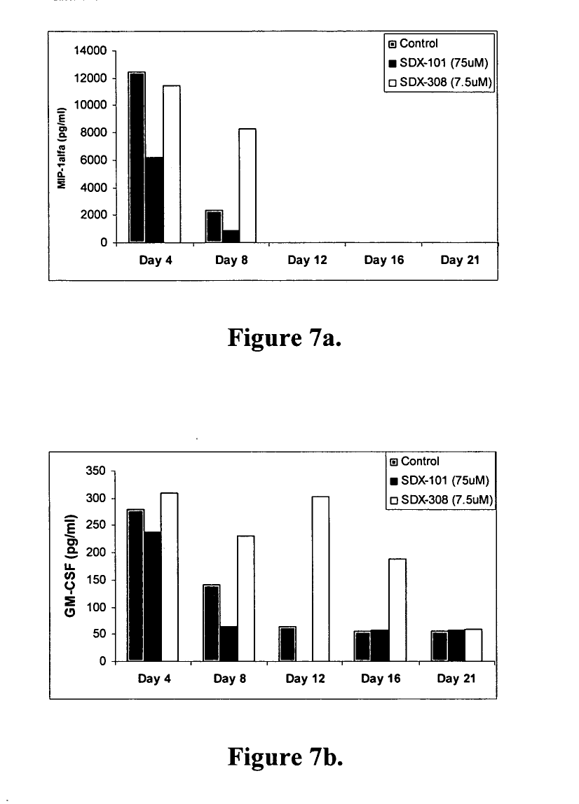

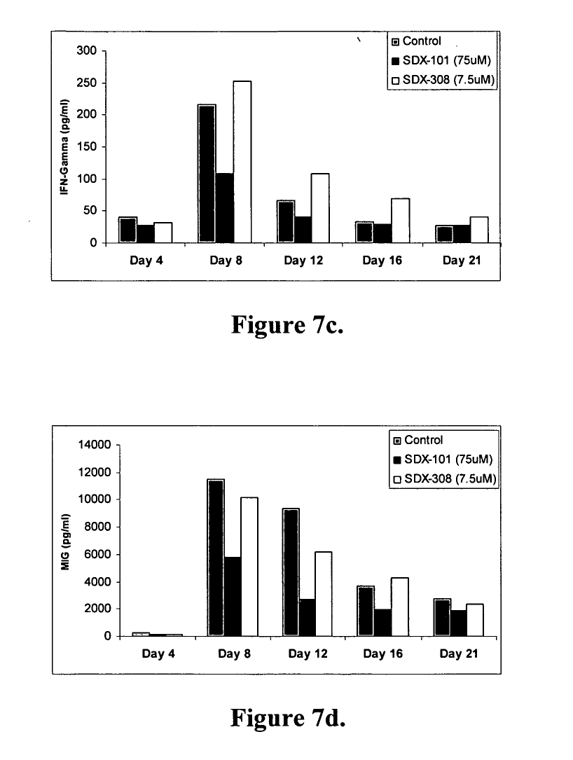

When treated with SDX-101, MIP-1α was significantly decreased to 6,000 pg/ML in comparison to controls of 12,000 pg/ML on day four. (FIG. 7A) Treatment with SDX-308 only slightly decreased MIP-1α secretion in osteoclast assays. Two other cytokines, IFN-γ and MIG (monokine induced by IFN-gamma) were also significantly decreased under the treatment with SDX-101 in osteoclast formation assays (FIGS. 7B and 7C). SDX-308 had less effects on MIG and IFN-γ cytokine secretion in osteoclast cultures (FIG. 7D). In contrast, GM-CSF, which also is known to inhibit osteoclastogenesis, was very strongly up-regulated by SDX-308 on day 12 (300 pg/ML) in comparison to control (55 pg/ML). These data suggest that SDX-101 and SDX-308 influence the cytokine secretion in a different pattern and therefore might act on osteoclastogenesis via different pathways.

### EXAMPLE 8

### SDX-308 Inhibits NF-κB Activation in OCL Precursors and MM Cells

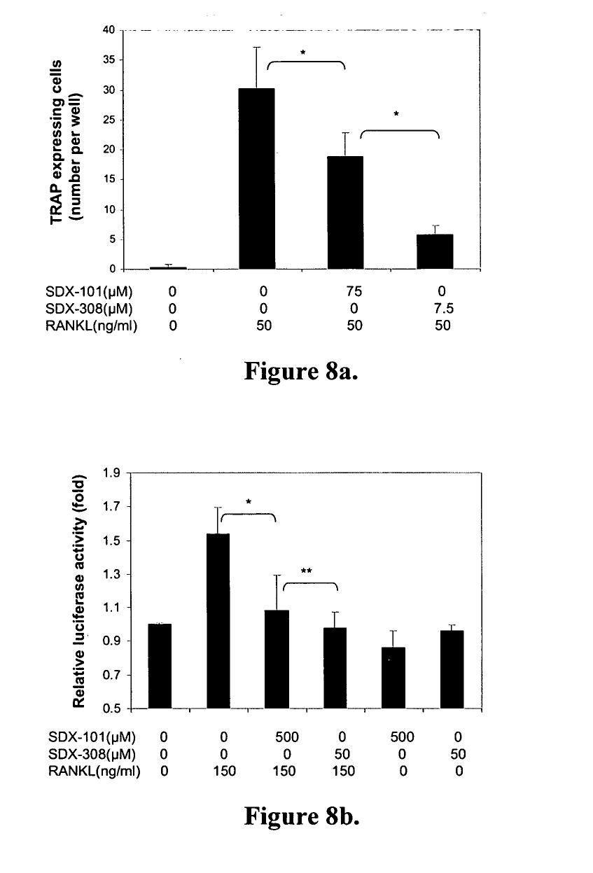

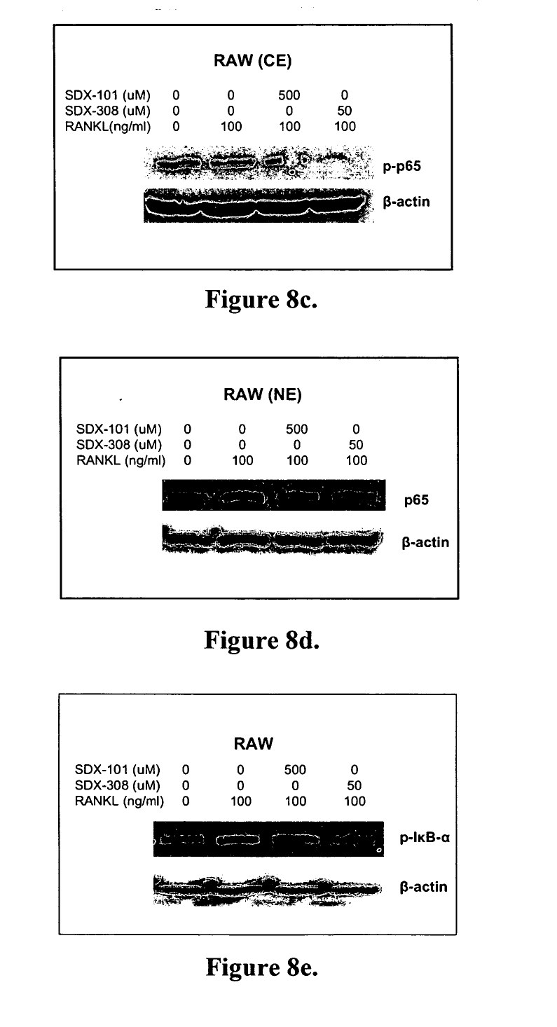

The transcription factor NF-κB plays a central role in OCL formation, and blocking NF-κB activity is a potential strategy for inhibition of OCL formation resulting in decreased bone resorption. To investigate the effects of SDX-101 and SDX-308 on the RANKL-induced activation of NF-κB as well as OCL formation, an osteoclast cellular model, RAW 264.7 cells, was used. The cells were incubated with SDX-101 or SDX-308 in the presence of RANKL and allowed to differentiate into OCLs after 5 days of culture. As shown in FIG. 8A, both SDX-101 and SDX-308 suppressed RANKL-induced OCL formation from RAW 264.7 cells. Again, SDX-308 at 7.5 μM induced a significantly stronger inhibition than SDX-101 at 75 μM. To examine the effects of SDX-101 and SDX-308 on RANKL-induced activation of NF-κB, RAW 264.7 cells transient transfected with a NF-κB luciferase reporter gene were used to test the effects of SDX-101 and SDX-308 on RANKL-stimulated luciferase activities. The harvested cell lysates were measured for luciferase activity. As shown in FIG. 8A, RANKL alone induced NF-κB activation, and treatment of SDX-101 or SDX-308 suppressed RANKL-induced NF-κB activation. SDX-101 and SDX-308 alone had no significant effects on the luciferase activity (FIG. 8B) and cellular viability (data not shown).

It is well known that once NF-κB is activated it translocates into the nucleus, where it binds to the appropriate response element sequences in the promoter regions of target genes. Therefore, whether SDX-101 and SDX-308 inhibit the phosphorylation and nuclear translocation of NF-κB p65 was investigated. Short incubation (30 minutes) of SDX-308 at a concentration of 50 μM displayed strong inhibition of constitutive and RANKL-induced p65 phosphorylation as well as NF-κB p65 nuclear translocation in OCL precursor RAW264.7 cells (FIGS. 8C and 8D). SDX-101 had only minor effects.

Because NF-κB activation by most stimulators requires phosphorylation and degradation of its inhibitory subunit IκBα, whether the inhibition of NF-κB activation by SDX-308 was due to the suppression of IκBα signaling was investigated. The data show that high levels of IκBα phosphorylation were observed in the OCL cell line RAW 264.7. Treatment of SDX-308 decreased constitutive and RANKL-induced phosphorylation level of IκBα (FIG. 8E).

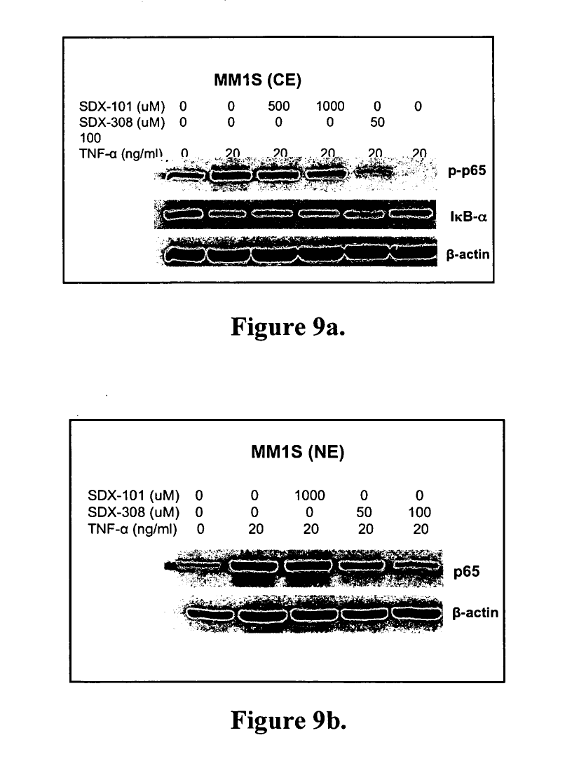

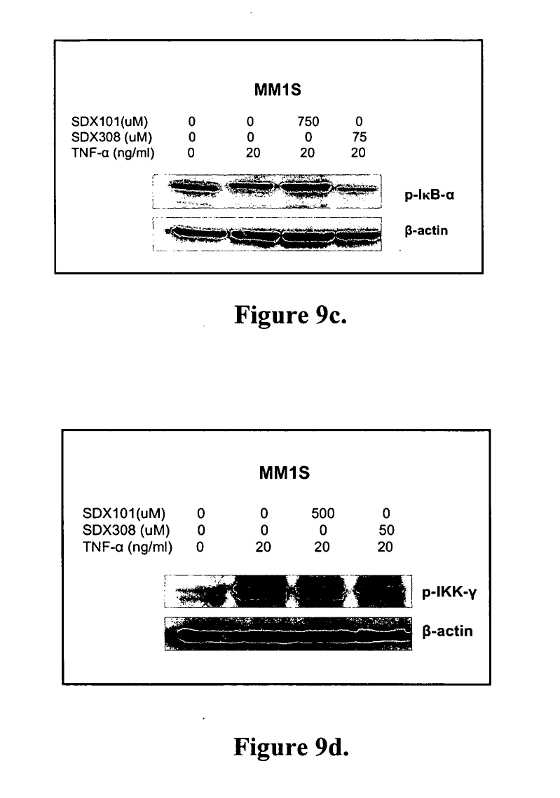

In another set of experiments, human MM cell line MM.1S cells were used to confirm the inhibitory effects of SDX compounds on NF-κB activation. TNFα stimulation for 15 minutes resulted in obvious phosphorylation and nuclear shift of NF-κB p65. However, pretreatment with SDX-308 significantly decreased TNF-induced p65 phosphorylation and nuclear translocation (FIGS. 9A and 9B). Furthermore, as shown in FIG. 9A, SDX-308 exposure also displayed strong inhibition of constitutive phosphorylation of p65. In addition, SDX-308 could inhibit TNF-induced IκBA phosphorylation and degradation, suggesting that SDX-308 might inhibit the release of NF-κB from IκBα (FIG. 9A middle panel and 9C). In contrast, SDX-101 had minor or no obvious effect on IκBα phosphorylation and degradation.

As the regulatory subunit of IκB kinase (IKK) complex, IKK-γ plays a central role in transporting IKK toward its activating kinase (e.g., NIK). Activation of IKK is required for TNF-induced phosphorylation and degradation of IκB and subsequent p65 activation (Yamamoto Y et al. (2001) J. Biol. Chem. 276:36327-36). Since SDX-308 inhibited IrB-p65 pathway, whether SDX-308 could attenuate the activation of IKK-γ was evaluated. As shown in FIG. 9D, SDX-308 pretreatment markedly decreased TNF-induced phosphorylation levels of IKK-γ in MM.1S cells, indicating the regulatory pathway of IKK-IκB was interrupted by SDX-308 treatment.

The present invention is not limited to the embodiments described and exemplified above, but is capable of variation and modification within the scope of the appended claims.

What is Claimed:

1. A method for treating osteolytic bone disease in a subject in need of such treatment, comprising administering to the subject a composition comprising a pharmaceutically acceptable carrier and at least one cyclooxygenase-2 (COX-2) inhibitor in an amount effective to treat osteolytic bone disease in the subject.

2. The method of claim 1, wherein the COX-2 inhibitor is SDX-101, S-etodolac, celecoxib, rofecoxib, valecoxib, lumiracoxib, eltoricoxib, or an analog, homolog, conjugate, or derivative thereof.

3. The method of claim 1, wherein the COX-2 inhibitor inhibits macrophage inflammatory protein 1-alpha (MIP-1α) expression.

4. The method of claim 1, wherein the osteolytic bone disease is multiple myeloma, osteoporosis, metastatic breast cancer, metastatic lung cancer, metastatic prostate cancer, infantile systemic hyalinosis, or infantile myofibromatosis.

5. The method of claim 1, wherein the subject is a mammal.

6. The method of claim 5, wherein the mammal is a human.

7. A method for treating osteolytic bone disease in a subject in need of such treatment, comprising administering to the subject a composition comprising a pharmaceutically acceptable carrier and at least one inhibitor of NF-κB activation in an amount effective to treat osteolytic bone disease in the subject.

8. The method of claim 7, wherein the inhibitor of NF-κB is SDX-308.

9. The method of claim 7, wherein the osteolytic bone disease is multiple myeloma, osteoporosis, metastatic breast cancer, metastatic lung cancer, metastatic prostate cancer, infantile systemic hyalinosis, or infantile myofibromatosis.

10. The method of claim 7, wherein the subject is a mammal.

11. The method of claim 10, wherein the mammal is a human.

12. A method for inhibiting osteoclastogenesis in a subject comprising administering to the subject a composition comprising a pharmaceutically acceptable carrier and at least one COX-2 inhibitor in an amount effective to inhibit osteoclastogenesis in the subject.

13. The method of claim 12, wherein the COX-2 inhibitor is SDX-101, S-etodolac, celecoxib, rofecoxib, valecoxib, lumiracoxib, eltoricoxib, or an analog, homolog, conjugate, or derivative thereof.

14. The method of claim 12, wherein the COX-2 inhibitor inhibits MIP-1α expression.

15. The method of claim 12, wherein the subject is a mammal.

16. The method of claim 15, wherein the mammal is a human.

17. A method of inhibiting MIP-1α in a subject comprising administering to the subject a composition comprising a pharmaceutically acceptable carrier and at least one cyclooxygenase-2 (COX-2) inhibitor in an amount effective to inhibit MIP-1α expression in the subject.

18. The method of claim 17, wherein the COX-2 inhibitor is SDX-101, S-etodolac, celecoxib, rofecoxib, valecoxib, lumiracoxib, eltoricoxib, or an analog, homolog, conjugate, or derivative thereof.

19. The method of claim 17, wherein the subject is a mammal.

20. The method of claim 19, wherein the mammal is a human.

21. A method for inhibiting osteoclastogenesis in a subject comprising administering to the subject a composition comprising a pharmaceutically acceptable carrier and at least one inhibitor of NF-κB activation in an amount effective to inhibit osteoclastogenesis in the subject.

22. The method of claim 21, wherein the inhibitor of NF-κB activation is SDX-308.

23. The method of claim 21, wherein the subject is a mammal.

24. The method of claim 23, wherein the mammal is human.
